(19) United States
(12) Patent Application Publication
(10) Pub. No.: US 2006/0088477 A1
(43) Pub. Date: Apr. 27, 2006

(54) Compositions and Methods of Dispensing Palliative or Therapeutic Agents

(75) Inventor: Gerald L. Maurer, Cincinnati, OH (US)

Correspondence Address: LAFKAS PATENT LLC, 7811 LAUREL AVENUE, CINCINNATI, OH, 45243, US

(73) Assignee: NATIONAL RESEARCH LABORATORIES, LTD., Cincinnati, OH (US)

(21) Appl. No.: 11/163,616

(22) Filed: Oct. 25, 2005

Related U.S. Application Data
(60) Provisional application No. 60/522,646, filed on Oct. 25, 2004.

(51) Int. Cl. A61L 9/04; A61K 31/315; A61K 31/30

(52) U.S. Cl. 424/45; 514/494; 514/499

(57) ABSTRACT

The present invention is a palliative or therapeutic foam for medicating or treating affected biological tissue having a pH of 7.0 to 8.0 and including a hydrated dialkali monometal complex such as disodium monocopper(II) citrate dihydrate or disodium monozinc(II) citrate dihydrate and triethanolamine lauryl sulfate in a concentration of less than 10%.

### CROSS-REFERENCE TO RELATED APPLICATION

This application for a patent claims priority to U.S. Provisional Patent Application No. 60/522,646 as filed Oct. 25, 2004.

### BACKGROUND

The various exemplary embodiments of the present invention relate generally to a composition and method of using the composition to palliate or treat affected biological tissues in mammals. More particularly, the various exemplary embodiments of the present invention relate to a method and a composition for treating damaged or affected biological tissue comprising one or more therapeutic agents and a foaming agent in a physiological pH range.

Inflammation is a local and protective response to tissue injury and destruction of cells. The precise elements constituting the inflammatory response vary according to the site of injury, the state of the body, and the injurious agent, such as bacteria or trauma. Should the inflammatory response become impaired or compromised, however, the corresponding tissue will undergo a degenerative process stimulating further injury and cell destruction. Obviously, then, the inflammatory response embodies a multifaceted process that is required to promote and rehabilitate normal tissue function. Therefore, since the inflammatory response is generally similar with various stimuli, it can be viewed and treated as a relatively nonspecific response.

Presently, conventional anti-inflammatory therapy includes application of heat, exercise, salicylates to tolerance, indomethacin or butazolidin, and oral and intra-articular steroids. The above anti-inflammatory protocol, however, is less than optimum because it provides only a means to inhibit some component of the inflammatory process in a generally temporary or transient fashion. In other words, it treats the symptoms rather than promoting tissue repair or alleviating the causes of the degeneration.

Currently there are known methods of treating inflammation of tissue with metals such as copper. For example, it has been known since ancient Egypt that copper has been indicated for therapeutically treating granulomatous inflammation. It has been well established that the dissolution of copper from copper jewelry, for example, bracelets, worn in contact with skin appears to have therapeutic anti-inflammatory effects. In other studies, subdermal copper implants in rats have been demonstrated to exhibit anti-inflammatory activity. In a further instance, a neutral copper (II) bis(glycine) complex perfused through cat skin demonstrating that skin is permeable to soluble copper. In still a further instance several oral and parenteral copper complexes have been somewhat successfully used in the treatment of inflammation or arthritis. Finally, dermally applied copper complexes have been confirmed as pharmacoactive anti-inflammatory agents.

Clearly, various prior art approaches have been taken to employ copper as a means to directly alleviate the causes of inflammation and to promote tissue repair, which has led to have led to several improved copper compositions and dosage forms in an effort to maximize delivery of copper to the inflammatory areas. Examples of such delivery systems of the copper include parenteral (subcutaneous, intravascular, or intramuscular injection), oral, topical or inserts. The parenteral delivery of copper may be painful, inconvenient, require the presence of a physician, and cause further irritation at the site of injection. The oral delivery, on the other hand, often results in poorly absorbed copper by the gastric lining, thereby reducing their anti-inflammatory activity. Finally, the topical delivery of copper is commonly used when selecting a route in medicating inflammation such as, for example, arthritis. The administration of such topical dosage forms are patently desirable because of their unique and advantageous characteristics.

Notwithstanding the notoriety for topical dosage forms, many past and present topical copper complexes have not performed to their anticipated expectations as a means to effectively and conveniently treat inflammation or arthritis with copper. For example, the application of metal salts to proteinaceous membranes, such as skin, results in the attachment of the copper ions to the membrane components to form copper proteinates or salts. Thus, little if any copper ion, in the soluble, ionized state is ever introduced into the targeted inflammatory, for example, arthritic, areas. Further, copper salts can be corrosive to the skin possibly causing the patient to incur various types of lytic reactions. To overcome this undesirable characteristic, copper ions are complexed with a ligand or chelant to form a metal complex. That is, the copper is shielded from binding to the membrane components. An example of such topical complexes include copper-amine complexes and copper EDTA. Unfortunately, there are undesirable characteristics associated with these complexes which obviate their usefulness.

In U.S. Pat. No. 4,680,309 to the same inventor as the present invention, it is taught that tissue inflammation may be alleviated by delivering a metal complex consisting of a dialaki metal monoheavy metal chelate of an alpha or beta-hydroxy polycarboxlic acid. An example of the metal complex given is dialkalimetal monocopper (II) citrate.

Zinc ions are well known to have anti-viral activity. For example, the salt known as zinc acetate is used as a control substance in evaluating anti-viral compounds because zinc acetate is very toxic to viruses. However, such zinc salts have two inherent disadvantages that make them useless as therapeutic agents. In particular, the zinc salt is quite toxic to normal cells and it is very acidic. This makes it unsuitable for application to skin, much less mucus membranes. Further, because it is so acidic, about a pH of about 5, the zinc of zinc acetate is converted into an insoluble zinc oxide that has little or no anti-viral activity.

What is desired, however, is a means of dispensing and treating affected biological tissue with a therapeutic compound having the therapeutic advantages of copper, zinc, or both, without the disadvantages of traditional solid, cream, gels, or liquid applications of compounds. It would also be desired to decrease the concentration of copper, zinc, or both in a therapeutic compound.

### SUMMARY

The various exemplary embodiments of the present invention include palliative or therapeutic foam for treating or medicating affected biological tissue. The foam is comprised of one or more therapeutic agents and a foaming agent in a physiological pH range.

The various exemplary embodiments of the present invention further include a method for treating or medicating affected biological tissue, comprising dispensing a palliative or therapeutic foam from a foam dispenser; and applying the palliative or therapeutic foam to the affected biological tissue. The palliative or therapeutic foam is at a pH of about 7.0 to less than about 8.0. The foam is comprised of a hydrated dialkali monometal polycarboxylate complex and triethanolamine lauryl sulfate (TEA-LS) in a concentration of about 10% or less.

### DETAIL DESCRIPTION

The various exemplary embodiments of the present invention include compositions and methods for applying palliative or therapeutic agents to biological tissues affected by problematic tissue conditions. The various exemplary compositions of the present invention have shown unrealized benefits in the treatment of some problematic tissue conditions. In particular, it has been most unexpectedly found that by dispersing palliative agents, therapeutic agents or a combination thereof in a liquid system that is subsequently dispensed in the form of a foam to the area of the affected biological tissues, small amounts of the said agents than needed in the liquid form of the same agents may be applied to obtain desired palliative or therapeutic effects.

Furthermore, as an additional benefit, it has been discovered that irritation of the affected biological tissues having attendant discomfort, pain or both is surprisingly reduced when employing the compositions and methods according to the exemplary embodiments of the present invention.

There is a further benefit in the compositions and methods according to the exemplary embodiments of the present invention. In particular, it has been found that the physical form of the agents according to the exemplary embodiments of the present invention in the high energy level of the foam itself offers delivery of the agents in a finely divided, miniscule form in contrast to a solid or liquid application of the same agents to the affected biological tissues.

The novel, unexpected and utilitarian benefits of the compositions and methods according to the exemplary embodiments of the present invention have found to be especially applicable to use in the oral cavity and to chemical, thermal or other types of radiation-damaged tissue as a skin for burn victims or therapeutic radiation irritation of mucous membranes associated with certain cancer therapies.

The various exemplary embodiments of the present invention comprises a palliative or therapeutic foam for treating or medicating effected biological tissue. This foam comprises one or more therapeutic agents and a foaming agent in physiological pH range.

In a preferred exemplary embodiment, the foaming agent is triethanolamine lauryl sulfate (TEA-LS). It is preferred that the TEA-LS is in a concentration of about 10% or less in the palliative or therapeutic foam.

In the various exemplary embodiments, it is preferred that the therapeutic agent comprise a hydrated dialkali monometal polycarboxylate 1:1 molar ratio of metal-to-complexing agent.

The metal-to-complexing agent is a multivalent metal and a polyfunctional organic ligand in a ratio of 1:1 of the metal to the ligand and has a dissociation property represented by a sigmoidally shaped plot on a pM-pH diagram. Specific examples of the metal complex are dialkali metal monocopper(II) citrates represented by disodium-, dipotassium- or dilithiummonocopper(II) citrate. These dialkali monocopper(II) citrates have a dissociation property represented by a sigmoidal plot, wherein the curve of two directions meet at a point within the pH range of about 7 to about 9. It has been established that these monocopper(II) complexes in basic media, on the order of about pH 9 to about 12, are very stable, i.e., have an effective stability constant, K_{eff}, of the order of about 10¹² to about 10¹³. However, K_{eff} of these monocopper(II) citrate complexes at a pH of about 7-9 are on the order of about 10⁵ to about 10¹². Therefore, at a pH of around 7, the effective stability constant of the monocopper(II) citrate complex is considerably lower (a thousand to a several hundreds of thousand times lower) and a significant free Cu⁺⁺ concentration is available for anti-inflammatory activity. For example, about 10% of the copper in the complex is in the ionized state at or about pH 7 while approximately 0.1% of the copper is ionized at or about pH 9.

Thus, it is to be understood that the anti-inflammatory complexes of this invention are sensitive to pH, and as the pH is lowered to or below about 7, copper ion is made more available. If tissue is intact, i.e., healthy without trauma, then there are few, if any, free endogenous reacting moieties to induce the dissociation of copper ions. If there is trauma caused by inflammation, then the copper ions are induced to dissociate and complex with the endogenous reacting moieties associated with such trauma, thereby reducing or alleviating the inflammation. In general, the complexes will then tend to dissociate over a pH range of about 3 to about 12. Above about pH 12, the complexes tend to be destroyed by the alkaline media, precipitating from the media as hydrous metal oxides. Below about pH 7, the instability of the metal complex results in high concentrations of the free Cu⁺⁺ upon demand, as explained to effect anti-inflammatory activities. At the pathological pH of about 7, below the skin, the controlled release is most effective. The complexes will preferably be dispersed in a vehicle to provide a composition having a pH of about 6.5 to about 9 for passage through the tissue upon typical administration to provide controlled release of the metal ions upon presentment of endogenous reacting moieties that are associated with inflammatory activities.

In accordance with this description and the presently preferred embodiment, it will become apparent that other metal complexes of polyfunctional organic ligands respond to the model of this invention where they exhibit the dissociation property characterized by a sigmoidal curve on a standard pM-pH diagram. For example, based upon the monometal-polyfunctional organic ligand complex of this invention, other metal ions of a monovalent or multivalent nature, specifically, divalent and polyvalent cations including zinc, nickel, chromium, bismuth, mercury, silver, cobalt, and other similar metallic or heavy metal cations may be employed. Other polyfunctional organic ligands may be substituted for the citric acid specifically exemplified by the preferred embodiment of this invention. Included among other polyfunctional ligands are the broader class of alpha or beta hydroxy polycarboxylic acids into which class the citric acid falls. Also, other functionally substituted acids such as alpha or beta amino, sulfhydro, phosphinol, etc., can be substituted in the molecular model of the metal complex of this invention and similar results can be achieved.

One particularly desirable metal complex in the 1:1 dialkali monometal polyfunctional organic ligand chelate family is disodium monocopper (II) citrate dihydrate, CAS Registry #65330-59-8. This material is sold under the tradename MCC™ by National Research Laboratories, Ltd. of Cincinnati, Ohio.

As set forth above and in the prior art, it is known that the use of compounds such as MCC and similar compounds singly may be used in a wide variety of utilities, including as antimicrobial agents.

Another exemplary metal complex is disodium monozinc (II) citrate dihydrate, sold under the tradename MZC™ by National Research Laboratories, Ltd. of Cincinnati, Ohio. MZC has been shown to have, in particular, dramatic anti-inflammatory performance.

The prior art teaches using a foam to treat biological tissue wounds, however, the prior art does not give regard to any optimum physiological properties associated with such foam treatment. Further, the prior art teaches using solutions in the foam that utilize sodium lauryl sulfate and other sodium salts of well known surfactants. Such solutions as taught in the prior art have pH in ranges of 8-9, and in some cases, even higher. Oral health, especially, is very sensitive to pH, of about 6 to about 8, and introducing a pH level higher than normal physiological pH levels to biological tissue could be detrimental to the health of the biological tissue.

Further, the hydrated dialkali monometal polycarboxylate 1:1 molar ratio of metal-to-complexing agent exemplified by disodium monocopper(II) citrate dihydrate (MCC) or disodium monzinc(II) citrate (MZC), is optimally reactive at or below the physiological pH range of about 7.5, from about 6 to about 8, contingent upon the tissue being treated. The use of the prior art to dispense MCC in any therapeutic formulation not possible and inoperative.

Surprisingly, using triethanolamine lauryl sulfate (TEA-LS) at a concentration of about 5% has a pH of only about 7.3 to about 7.4. This pH of the TEA-LS is an ideal range for use with MCC, MZC, or both, which are active ingredients in many therapeutic mixtures. TEA-LS is gentle to sensitive oral tissues as opposed to the nearly caustic properties of the prior art compositions.

The surfactants of the prior art are also only usable orally to a concentration of about 5% due to taste considerations. In the exemplary embodiments of the present invention, a concentration of about 10% TEA-LS in the composition is essentially tasteless, thereby allowing for greater concentrations and therefore, longer-lasting and more stable foams to be generated and applied.

Longer-lasting and more stable foams as in the present invention are highly favored for the delivery of drugs and the like to affected surfaces due to their increased surface areas being able to last longer and, therefore, allowing more intimate contact of the finely dispersed mendicant with the affected biological tissues.

The various exemplary embodiments of the present invention includes a method for treating or medicating affected biological tissue, comprising dispensing a palliative or therapeutic foam from a foam dispenser; and applying the palliative or therapeutic foam to the affected biological tissue. Exemplary embodiments teach that the palliative or therapeutic foam comprises MCC, MZC, or both in addition to triethanolamine lauryl sulfate (TEA-LS) in a concentration of about 10% or less. In various exemplary embodiments, the foam is at a pH of about 7.0 to less than about 8.0.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

What is claimed is:

1. A palliative or therapeutic foam for treating or medicating affected biological tissue, comprising:
one or more therapeutic agents; and
a foaming agent in a physiological pH range.

2. The foam according to claim 1, wherein the foaming agent is triethanolamine lauryl sulfate (TEA-LS) at a concentration of about 10% or less.

3. The foam according to claim 2, wherein the TEA-LS is at a concentration of about 5%.

4. The foam according to claim 1, wherein the foaming agent is at a pH of about 7.3 to about 7.4.

5. The foam according to claim 1, wherein the therapeutic agent is a hydrated dialkali monometal polycarboxylate complex comprised of a multivalent metal and at least one polyfunctional organic ligand, wherein the ligand is in the form of an alkaline earth salt and a molar ratio of metal to ligand is 1:1.

6. The foam according to claim 5, wherein the multivalent metal is selected from the group consisting of copper, zinc, nickel, chromium, bismuth, mercury, silver, and cobalt.

7. The foam according to claim 5, wherein the monometal complex is disodium monocopper (II) citrate dihydrate (MCC).

8. The foam according to claim 5, wherein the monometal complex is disodium monozinc (II) citrate dihydrate (MZC).

9. The foam according to claim 1, the foam has a pH of about 7.0 to less than about 8.0.

10. A method for treating or medicating affected biological tissue, comprising:
dispensing a palliative or therapeutic foam from a foam dispenser; and
applying the palliative or therapeutic foam to the affected biological tissue;
wherein the palliative or therapeutic foam is at a pH of about 7.0 to less than about 8.0 and comprises:
a hydrated dialkali monometal polycarboxylate complex; and
triethanolamine lauryl sulfate (TEA-LS) in a concentration of about 10% or less.

11. The method according to claim 10, wherein the concentration of TEA-LS is about 5%.

12. The method according to claim 10, wherein the pH of TEA-LS is about 7.3 to about 7.4.

13. The method according to claim 10, wherein the complex is comprised of a multivalent metal and at least one polyfunctional organic ligand, wherein the ligand is in the form of an alkaline earth salt and a molar ratio of metal to ligand is 1:1.

14. The method according to claim 13, wherein the the multivalent metal is selected from the group consisting of copper, zinc, nickel, chromium, bismuth, mercury, silver, and cobalt.

15. The method according to claim 13, wherein the monometal complex is disodium monocopper (II) citrate dihydrate (MCC).

16. The method according to claim 13, wherein the monometal complex is disodium monozinc (II) citrate dihydrate (MZC).

17. The method according to claim 10, wherein the affected tissue is an oral cavity.

18. The method according to claim 10, wherein the affected tissue is tissue damaged by chemical, thermal, radiation, or combination thereof.
